(19) United States
(12) Patent Application Publication
(10) Pub. No.: US 2020/0163812 A1
(43) Pub. Date: May 28, 2020

(54) ABSORBENT PANTS HAVING A WAISTBAND WITH A REAR POCKET

(71) Applicant: Kimberly-Clark Worldwide, Inc., Neenah, WI (US)

(72) Inventors: Roxanne M. Zuleger, Appleton, WI (US); Sarah Kleuskens, Neenah, WI (US); Jessica Andriessen, Appleton, WI (US); Dan Cote, Appleton, WI (US)

(21) Appl. No.: 16/611,285

(22) PCT Filed: Jan. 31, 2018
(86) PCT No.: PCT/US2018/016092

§ 371 Date: Nov. 6, 2019

(51) Int. Cl. A61F 13/494; A61F 13/49; A61F 13/496

(52) U.S. Cl. CPC A61F 13/49466; A61F 13/49011; A61F 2013/588; A61F 2013/49493; A61F 13/496

(57) ABSTRACT

An absorbent garment that includes a longitudinal axis and a lateral axis. The absorbent garment is a pant that includes a front region, a back region and a crotch region, with the crotch region being disposed between the front region and the back region. The crotch region is formed by an absorbent assembly. In addition to the absorbent assembly, the garment includes a front panel and a back panel; the front panel is attached to a front region of the absorbent assembly and the back panel is attached to a back region of the absorbent assembly. The front panel and the back panel are bonded to each other to form the lateral side seams of the garment. The garment also includes a pocket at the back waist of the pant that is formed, in part, by a folded over portion of the back panel. The pocket includes a fluid barrier.

### BACKGROUND

Absorbent articles, such as diapers, incontinence garments, training pants, sanitary napkins, panty liners, and the like are well known in the art. These articles, which are often disposable, are capable of absorbing and retaining fluids and other bodily discharges. Some absorbent articles, such as pull-on type absorbent articles, include a central absorbent member and side panels extending from and interconnecting respective front and back regions of the absorbent member forming a waist assembly.

An example of a pull-on absorbent article is a diaper pant; diaper pants are typically worn by babies who are able to stand up, but who are not yet old enough to be toilet trained. Absorbent articles in the diaper pants format typically include a waist assembly (that includes side panels) formed of elastic strands or other elastic materials placed between inner and outer layers of a nonwoven material. The elastic strands are typically kept in place by adhesive. These types of waist assemblies may include 20 to 30 high-tension elastic strands that embrace the waist area. The use of the elastic strands in the side panels of the diaper pants allows the side panels to be highly elastic and allows for a wide range of body shapes over which the diaper pant may be donned. The elastic side panels also provide a conforming fit for the wearer.

Traditional diaper pants typically lack structural features that impede the movement of bowel movements, particularly more fluid bowel movements. As a result, bowel movements may travel outside of the diaper pant, such as out through the rear waist opening. Insufficiency at containing bowel movements will depend on many factors, including individual anatomy, consistency of the bowel movement, and position of the child when the bowel movement occurs. A desirable feature of a diaper pant is to have a soft and comfortable waistband or waist area—one that does not put undue pressure on the skin, which could lead to red-marking of the skin. However, softer and more comfortable waistbands/waist areas are not necessarily conducive to containing bowel movements within the diaper pant.

There remains a need for a diaper pant having improved capability to contain bowel movements, regardless of child anatomy, child position or bowel movement consistency. Further, there remains a need to provide such a containment benefit while not interfering with the function of the waistband and the comfort of the pant during wear. Any bowel movement containment solutions should not interfere with the waistband's ability to provide a “custom” fit to the wearer of the pant and such solutions should be consistent with the pant being soft and comfortable during wear. There also remains a need for a containment solution that does not affect how the diaper pant is donned on a wearer or how the diaper pant is removed.

### SUMMARY

The present invention is directed to an absorbent garment that provides a solution for improved containment of bowel movements within the garment. The product improvement does not interfere with the function or comfort of the waistband during use and the improvement does not interfere with the waistband providing a “custom fit”. Further, the improvement does not interfere with how the garment is donned or removed. The absorbent garments of the invention have a longitudinal direction and a lateral direction. The lateral direction is generally perpendicular to the longitudinal direction and describing the absorbent garments with these orientations improves the understanding of the components of the garment in relation to one another. The absorbent garments include a front region, a back region and a crotch region—where the crotch region is disposed between the front region and the back region. The crotch region includes an absorbent assembly. The absorbent assembly has a front edge, a back edge and a pair of laterally-opposed side edges (that is, side edges that are opposite each other in the lateral direction). The absorbent assembly includes a skin-facing liner, an absorbent composite and an outer cover. The outer cover includes a liquid impermeably layer that prevents bodily fluids from passing out of or leaking out of the absorbent assembly. The absorbent garments also include a front panel in the front region and a back panel in the back region. Both the front panel and the back panel include an inner layer, an outer layer and a stranded elastic area that is disposed between the inner layer and the outer layer. The front panel and the back panel are bonded or otherwise joined together to form side seams of the absorbent garment. The improvement of absorbent garments of the invention over other absorbent garments is that a portion of the back panel is folded over the back edge of the absorbent assembly and forms a fold line. The folded over portion of the back panel includes at least one elastic strand and there is a fluid barrier between the folded over portion and the skin-facing liner of the absorbent assembly. The folded over portion of the back panel forms a “pocket” to trap and contain bodily wastes. The fluid barrier between the folded over portion of the back panel and the skin-facing liner helps ensure fluids do not leak through the material from which the back panel is formed.

The fluid barrier between the folded over portion of the back panel and the skin-facing liner may be formed in different ways. In some aspects of the present invention, the folded over portion of the back panel may be attached to the skin-facing liner. For example, the folded over portion of the back panel may be attached to the skin-facing liner proximate (meaning near) and along each of the laterally-opposed side edges of the absorbent assembly. The width of attachment in the lateral direction of the garment can be selected as appropriate. The means of attachment may be by adhesive or bonding (ultrasonic, pressure or thermal)—or other known means of attaching nonwoven materials to each other. Attachment of the folded over portion of the back panel to the skin-facing liner along the side edges of the absorbent assembly may form a part of the fluid barrier (particularly if the attachment means prevents the flow of fluid). The fluid barrier may be formed by a bead of adhesive between the folded over portion of the back panel and the skin-facing liner along the back edge of the absorbent composite. The bead of adhesive along the line of the back edge of the absorbent composite (and, typically, the absorbent assembly at the same time) prevents the flow/leakage of fluid through the fold line. The bead of adhesive along the back edge of the absorbent composite in combination with the attachment of the folded over portion of the back panel to the skin-facing liner along the side edges of the absorbent assembly may form the fluid barrier of the absorbent garment. In another aspect, the fluid barrier may be formed by a film layer of material that is attached to the folded over portion of the back panel. The film layer may be attached to the side/portion of the folded over portion of the back panel that faces away from the wearer's skin and faces toward the skin-facing liner. The film layer material may be selected from the film materials used as a component of the outer cover of the absorbent garment.

The absorbent garments of the invention may include a pair of containment flaps that extend along the length of the absorbent assembly in the longitudinal direction. Each containment flap may extend along one of the laterally-opposed side edges of the absorbent assembly. With executions of the absorbent garments of the invention including containment flaps, the folded over portion of the back panel may be attached to the skin-facing liner of the absorbent assembly by contacting the lines of the containment flaps. The folded over portion of the back panel may be attached to each of the containment flaps by adhesive or by bonding (ultrasonic, pressure or thermal).

In another aspect, the present invention is directed to an absorbent garment having a longitudinal direction and a lateral direction. The lateral direction is generally perpendicular to the longitudinal direction and describing the absorbent garments with these orientations improves the understanding of the components of the garment in relation to one another. The absorbent garment includes a front region, a back region and a crotch region—where the crotch region is disposed between the front region and the back region. The crotch region includes an absorbent assembly. The absorbent assembly has a front edge and a pair of laterally-opposed side edges (that is, side edges that are opposite each other in the lateral direction). The absorbent assembly includes a skin-facing liner, an absorbent composite and an outer cover. The outer cover includes a liquid impermeably layer that prevents bodily fluids from passing out of or leaking out of the absorbent assembly. The skin-facing liner and the outer cover extend beyond a back edge of the absorbent composite. With this aspect of the invention, the back edge of the absorbent composite and the back edge of the absorbent assembly are the same edge. The absorbent garments also include a front panel in the front region and a back panel in the back region. Both the front panel and the back panel include an inner layer, an outer layer and a stranded elastic area that is disposed between the inner layer and the outer layer. The front panel and the back panel are bonded or otherwise joined together to form side seams of the absorbent garment. As with the other executions of the absorbent garment of the invention, a portion of the back panel is folded over the back edge of the absorbent composite/absorbent assembly and forms a fold line. The folded over portion of the back panel includes at least one elastic strand. The portions of the skin-facing liner and the outer cover that extend beyond the back edge of the absorbent composite, extend in parallel with the folded over portion of the back panel. For the portions of the skin-facing liner, the outer cover and the folded over portion of the back panel that overlap each other, they may be attached to each other by adhesive or bonding (ultrasonic, pressure or thermal), so that there is not open space in between them. Further with regard to the folded over portion of the back panel that extends in parallel with the skin-facing liner and the outer cover, the combination of the three materials ends (terminates) at an edge of the folded over portion. The edge of the folded over portion does not reach the back edge of the absorbent composite. A purpose of the edge of the folded over portion not reaching the back edge of the absorbent composite is so that the back waist pocket is formed to receive bodily wastes and prevent them from leaking out of the absorbent garment. More particularly, the distance between the edge of the folded over portion and the back edge of the absorbent composite is greater than five (5) millimeters. This distance may be selected to improve the function of the back waist pocket.

With this aspect of the invention, the absorbent garment may include a pair of containment flaps that extend along the length of the absorbent assembly in the longitudinal direction. Each containment flap may extend along one of the laterally-opposed side edges of the absorbent assembly. With executions of the absorbent garments of the invention including containment flaps, the folded over portion of the back panel may be attached to the skin-facing liner of the absorbent assembly by contacting the lines of the containment flaps. The folded over portion of the back panel may be attached to each of the containment flaps by adhesive or by bonding (ultrasonic, pressure or thermal).

### DESCRIPTION OF THE DRAWINGS

The present disclosure will be more fully understood, and further features will become apparent, when reference is made to the following detailed description and the accompanying drawings. The drawings are merely representative and are not intended to limit the scope of the claims.

FIG. 1 is a top down view of an absorbent garment of the present disclosure, in an unassembled condition;

FIG. 2 is a front view of the absorbent garment of FIG. 1, in an assembled condition;

FIG. 3 is a top down view of the back region of an absorbent garment of the present disclosure in an unassembled condition;

FIG. 4 is a cross-sectional view of an aspect of the back region of an absorbent garment of the present disclosure;

FIG. 5 is a cross-sectional view of another aspect of the back region of an absorbent garment of the present disclosure; and

FIG. 6 is a cross-sectional view of a further aspect of the back region of an absorbent garment of the present disclosure.

Repeat use of reference characters in the present specification and drawings is intended to represent the same or analogous features or elements of the present disclosure. The drawings are representational and are not necessarily drawn to scale. Certain proportions thereof might be exaggerated, while others might be minimized.

### DETAILED DESCRIPTION

The term “absorbent garment” refers herein to a garment that can be placed against or in proximity to the body (i.e., contiguous with the body) of the wearer to absorb and contain various liquid, solid, and semi-solid exudates discharged from the body. Such absorbent articles, as described herein, are intended to be discarded after a limited period of use instead of being laundered or otherwise restored for reuse. It is to be understood that the present disclosure is applicable to various disposable absorbent garments, including, but not limited to, infant diaper pants, toilet-training pants, youth pants, swim pants, feminine hygiene garments and adult incontinence garments.

The term “bonded” refers herein to the joining, adhering, connecting, attaching, or the like, of two elements. Two elements will be considered bonded together when they are joined, adhered, connected, attached, or the like, directly to one another or indirectly to one another, such as when each is directly bonded to intermediate elements.

The term “carded web” refers herein to a web containing natural or synthetic staple fibers typically having fiber lengths less than about 100 mm. Bales of staple fibers can undergo an opening process to separate the fibers that are then sent to a carding process that separates and combs the fibers to align them in the machine direction after which the fibers are deposited onto a moving wire for further processing. Such webs are usually subjected to some type of bonding process such as thermal bonding using heat and/or pressure. In addition to or in lieu thereof, the fibers can be subject to adhesive processes to bind the fibers together such as by the use of powder adhesives. The carded web can be subjected to fluid entangling, such as hydroentangling, to further intertwine the fibers and thereby improve the integrity of the carded web. Carded webs, due to the fiber alignment in the machine direction, once bonded, will typically have more machine direction strength than cross machine direction strength.

The term “film” refers herein to a thermoplastic film made using an extrusion and/or forming process, such as a cast film or blown film extrusion process. The term includes apertured films, slit films, and other porous films that constitute liquid transfer films, as well as films that do not transfer fluids, such as, but not limited to, barrier films, filled films, breathable films, and oriented films.

The term “liquid impermeable” refers herein to a layer or multi-layer laminate in which liquid body exudates, such as urine, will not pass through the layer or laminate, under ordinary use conditions, in a direction generally perpendicular to the plane of the layer or laminate at the point of liquid contact.

The term “liquid permeable” refers herein to any material that is not liquid impermeable.

The term “meltblown” refers herein to fibers formed by extruding a molten thermoplastic material through a plurality of fine, usually circular, die capillaries as molten threads or filaments into converging high velocity heated gas (e.g., air) streams that attenuate the filaments of molten thermoplastic material to reduce their diameter, which can be a microfiber diameter. Thereafter, the meltblown fibers are carried by the high velocity gas stream and are deposited on a collecting surface to form a web of randomly dispersed meltblown fibers. Such a process is disclosed, for example, in U.S. Pat. No. 3,849,241 to Butin et al., which is incorporated herein by reference. Meltblown fibers are microfibers that can be continuous or discontinuous, are generally smaller than about 0.6 denier, and can be tacky and self-bonding when deposited onto a collecting surface.

The term “nonwoven” refers herein to materials and webs of material that are formed without the aid of a textile weaving or knitting process. The materials and webs of materials can have a structure of individual fibers, filaments, or threads (collectively referred to as “fibers”) that can be interlaid, but not in an identifiable manner as in a knitted fabric. Nonwoven materials or webs can be formed from many processes such as, but not limited to, meltblowing processes, spunbonding processes, carded web processes, etc.

The term “pliable” refers herein to materials that are compliant and that will readily conform to the general shape and contours of the wearer's body.

The term “spunbond” refers herein to small diameter fibers that are formed by extruding molten thermoplastic material as filaments from a plurality of fine capillaries of a spinnerette having a circular or other configuration, with the diameter of the extruded filaments then being rapidly reduced by a conventional process such as, for example, eductive drawing, and processes that described in U.S. Pat. No. 4,340,563 to Appel et al., U.S. Pat. No. 3,692,618 to Dorschner et al., U.S. Pat. No. 3,802,817 to Matsuki et al., U.S. Pat. Nos. 3,338,992 and 3,341,394 to Kinney, U.S. Pat. No. 3,502,763 to Hartmann, U.S. Pat. No. 3,502,538 to Peterson, and U.S. Pat. No. 3,542,615 to Dobo et al., each of which is incorporated herein in its entirety by reference. Spunbond fibers are generally continuous and often have average deniers larger than about 0.3, and in an aspect, between about 0.6, 5 and 10 and about 15, 20 and 40. Spunbond fibers are generally not tacky when they are deposited on a collecting surface.

The term “superabsorbent” refers herein to a water-swellable, water-insoluble organic or inorganic material capable, under the most favorable conditions, of absorbing at least about 15 times its weight and, in an aspect, at least about 30 times its weight, in an aqueous solution containing 0.9 weight percent sodium chloride. The superabsorbent materials can be natural, synthetic and modified natural polymers and materials. In addition, the superabsorbent materials can be inorganic materials, such as silica gels, or organic compounds, such as cross-linked polymers.

The term “thermoplastic” refers herein to a polymeric material that becomes pliable or moldable above a specific temperature and returns to a solid state upon cooling.

Generally, an absorbent garment having a novel structure is disclosed herein. The primary purpose of absorbent garments is to contain bodily waste (both fluid and solid). Pant format absorbent garments—such as those of the present disclosure—are typically designed and constructed to fit a wide range of body shapes and to be comfortable for the user to wear. A common pant construction includes a generally rectangular absorbent assembly integrated with a front waist panel and a back waist panel. The waist panels typically include stranded elastics and it is the working combination of the front panel and the back panel that provides the fit range of the pant. With the absorbent pant garments of the present disclosure, the lateral direction edges of the front panel and the back panel are bonded together to form the side seams of the garment. The novel feature of the absorbent pant garments of the present disclosure is that they include a “pocket” in the back region of the pant that is designed to contain mess and to prevent the mess from escaping the pant. The “pocket”—formed by a folded arrangement of garment materials—may be constructed in a variety of ways. The “pocket” includes a liquid barrier to prevent the mess from soaking through the materials used to construct the garment.

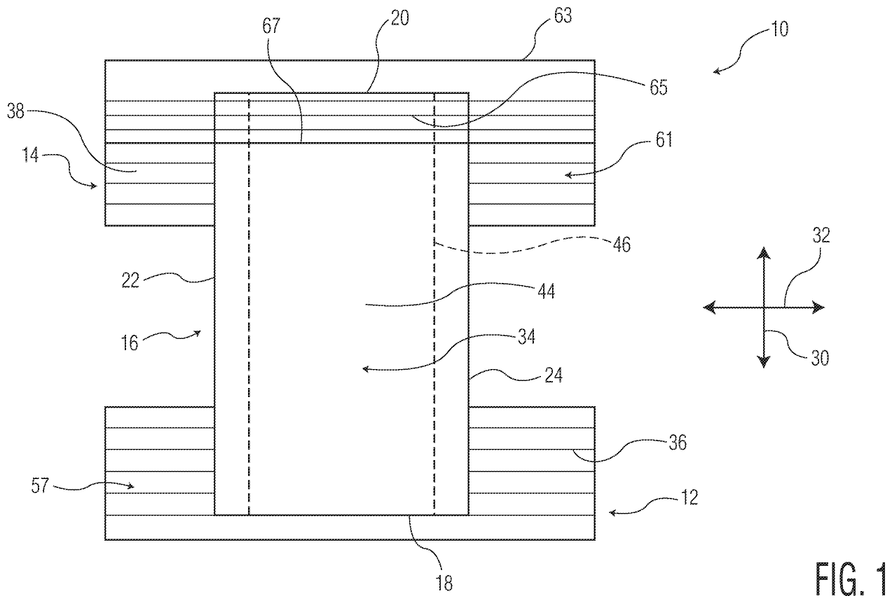

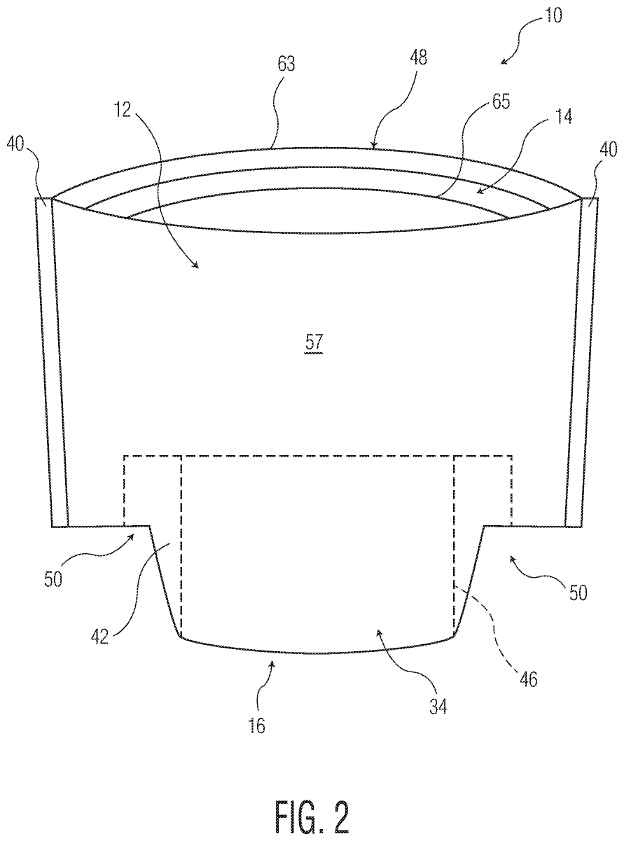

With reference to the drawings, FIGS. 1 and 2 illustrate one suitable aspect of an absorbent garment of the present disclosure in the form of a diaper pant, indicated generally at 10. While the present disclosure will be made in the context of the diaper pant 10, it should be understood that aspects of the present disclosure are applicable to other absorbent garments, such as, for example, toilet-training pants, youth pants, swim pants, feminine hygiene garments and adult incontinence garments. As used herein, the term “absorbent garment” refers to garments that absorb and contain body exudates and that are intended to be discarded after a limited period of use. The garments are not intended to be laundered or otherwise restored for reuse. Put differently, the garments are disposable. The garments can be placed against or in proximity to the body of a wearer to absorb and contain various exudates discharged from the body.

FIG. 1 illustrates the absorbent garment, such as a diaper pant, 10 in an unfolded and laid flat condition to show an inner surface of the absorbent garment that faces the wearer when the absorbent garment 10 is worn. The absorbent garment 10 has a longitudinal axis (direction) 30 and a lateral axis (direction) 32. Along the longitudinal axis 30, the absorbent garment 10 defines a front region 12, a back region 14, and a crotch region 16 extending between and connecting the front region 12 and the back region 14.

The absorbent garment 10 includes a central absorbent assembly, indicated generally at 34, that extends longitudinally from the front region 12 through the crotch region 16 to the back region 14. The central absorbent assembly 34 of the illustrated aspect includes an outer cover 42 (shown in FIG. 2) and a skin-facing (or “bodyside”) liner 44 connected to the outer cover 42 in a superposed relation by suitable means such as adhesives, ultrasonic bonds, thermal bonds, pressure bonds, or combinations thereof. An absorbent composite 46 is disposed between the outer cover 42 and the bodyside liner 44. The absorbent assembly 34 also includes a front edge 18, a back edge 20 and a pair of laterally-opposed side edges 22, 24.

The front and back regions 12, 14 of the absorbent garment 10 include a front panel 57 and a back panel, 61, respectively. The front panel 57 and the back panel 61 are interconnected via the absorbent assembly 34. The front panel 57 is a laminate or composite of several materials including an inner layer and an outer layer; the inner layer is closest to the skin of the wearer of the absorbent garment 10. Similarly, the back panel 61 is also a laminate or composite of several materials including an inner layer and an outer layer. Between the inner layer and outer layer of the front panel 57, there is a stranded elastic area 36. Between the inner layer and the outer layer of the back panel 61, there is a stranded elastic area 38. With respect to the back region 14 of the absorbent garment 10, a portion of the back panel 61 is folded over the back edge 20 of the absorbent assembly 34 forming a fold line 63. The folded over portion of the back panel 61 includes at least one elastic strand 65 and defines an edge 67. As will be described in more detail herein, there is a fluid barrier between the folded over portion of the back panel 61 and the skin-facing liner 44 of the absorbent assembly 34.

As shown in FIG. 1, the front panel 57 includes a pair of laterally-opposed, front side portions extending outward from the lateral center of the absorbent garment 10. Likewise, the back panel 61 includes a pair of laterally-opposed, back side portions extending outward from the lateral center of the absorbent garment 10. As shown in FIG. 2, the front panel 57 is joined to the back panel 61 by a pair of butt (or fin) side seams 40 to define a pull-on, pant-like configuration of the absorbent garment 10 having a waist opening, indicated at 48, and two leg openings, indicated at 50. More specifically, each front side portion is joined to a respective back side portion via one of the butt side seams 40.

With the absorbent garment 10 in the pull-on, pant-like configuration, illustrated in FIG. 2, the front region 12 includes the portion of the absorbent garment 10 that, when worn, is positioned at least in part on the front of the wearer while the back region 14 includes the portion of the absorbent garment 10 that is positioned at least in part on the back of the wearer. The crotch region 16 of the absorbent garment 10 includes the portion of the absorbent garment 10 that is positioned between the legs of the wearer and covers the lower torso of the wearer. The front and back side portions include the portions of the absorbent garment 10 (and more particularly of the front and back regions 12, 14) that, when worn, are positioned on the hips of the wearer. As shown in FIG. 2, the front panel 57 and the back panel 61 cooperatively define the waist opening 48 of the absorbent garment 10 that is configured to fully encircle the waist of the wearer.

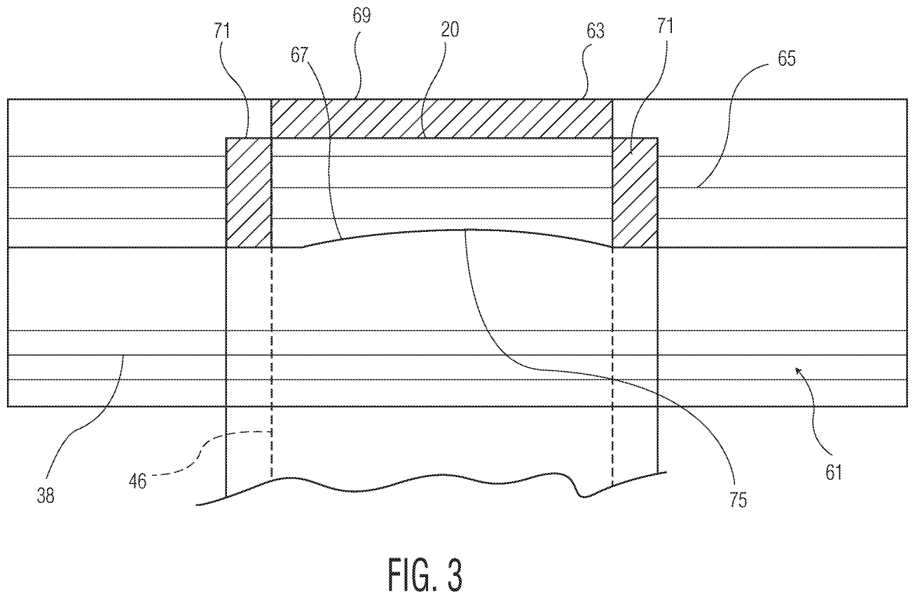

In one aspect of the present invention, and as illustrated in FIG. 3, the absorbent garment 10 includes a portion of the back panel 61 being folded over the back edge 20 of the absorbent assembly 34. Between the fold line 63 and the edge of the folded over portion 67, a back waist pocket 75 is formed. A purpose of the back waist pocket 75 in the absorbent garment 10 is to act as a barrier and to contain bodily wastes. In order to perform these functions, the back waist pocket 75 desirably includes a fluid barrier between the folded over portion of the back panel 61 and the skin-facing liner 44 of the absorbent assembly 34. The fluid barrier may be achieved in different ways. With the aspect shown in FIG. 3, the fluid barrier is achieved with a combination of lateral attachment areas 71 and a sealing bead of adhesive 69. The lateral attachment areas 71 are attaching the area of the folded over portion of the back panel 61 between the lateral edge of the absorbent composite 46 and the lateral edge of the absorbent assembly to the skin-facing liner 44 of the absorbent assembly 34. Therefore, the folded over portion of the back panel 61 is attached to the skin-facing liner 44 of the absorbent assembly 34 in a manner that is proximate and along each of the laterally-opposed side edges 22, 24 of the absorbent assembly 34. The lateral attachment areas 71 may be formed with adhesive or other suitable methods of attaching/bonding nonwoven materials together (including pressure bonding, thermal bonding and ultrasonic bonding). The sealing bead of adhesive 69 is located along the back edge 20 of the absorbent composite 46 and the fold line 63 of the folded over portion of the back panel 61. The combination of the lateral attachment areas 71 and the sealing bead of adhesive 69 creates a three-sided fluid barrier to contain bodily waste within the back waist pocket 75. In another aspect, the fluid barrier is formed by a film layer that is attached and part of the folded over portion of the back panel 61. The film layer may be formed of the same type of film material used to form the liquid impermeable layer of the outer cover 42. Executions of the absorbent garments 10 of the invention may include the absorbent assembly 34 having a pair of containment flaps (containment flaps are not shown in the Figures). Typically, the containment flaps are attached to the skin-facing liner 44 of the absorbent assembly 34. The containment flaps typically extend along the length of the absorbent assembly 34 in the longitudinal direction 30. More specifically, each containment flap would extend along and proximate one of the laterally-opposed side edges 22, 24 of the absorbent assembly 34. When containment flaps are incorporated into the absorbent garment 10, the folded over portion of the back panel 61 may be attached to the skin-facing liner 44 along contact lines with each of the containment flaps. The attachment to the skin-facing liner 44 via attachment to the containment flaps may occur by adhesive or other known means of attachment. Attachment via the containment flaps forms a portion of the fluid barrier.

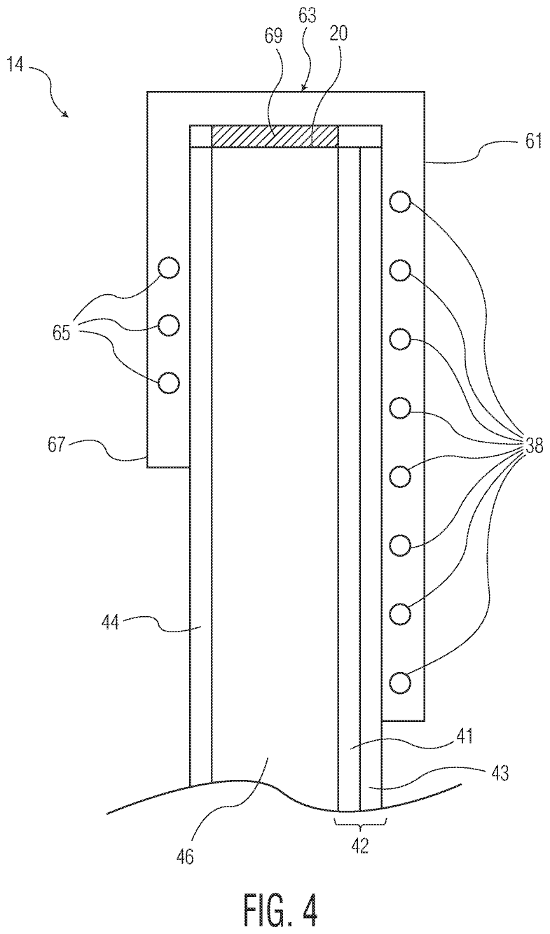

FIG. 4 representatively shows a cross-section of the back region 14 of the absorbent garment 10, including the back panel 61 in relationship to the absorbent assembly 34 and its components: skin-facing liner 44; absorbent composite 46; and outer cover 42. As is shown in the Figure, the outer cover 42 of absorbent garments 10 may be formed of two or more layers of materials. In this representation, the outer cover 42 is formed of a film layer 41 and an outer cover facing 43. The representation of FIG. 4 depicts how the back panel 61 of the absorbent garment 10 folds over the back edge 20 of the absorbent assembly 34—which is coterminous with the back edge 20 of the absorbent composite 46, in this example. The stranded elastic area 38 of the back panel 61 is shown in FIG. 4 as is the at least one stranded elastic 65 in the folded over portion of the back panel 61. The fold line 63 and the edge of the folded over portion 67 are also shown in FIG. 4. The back waist pocket 75 (not marked in FIG. 4) is formed between the folded over portion of the back panel 61 and the skin-facing liner 44. An element of a representative fluid barrier between the folded over portion of the back panel 61 and the skin-facing liner 44 is shown in FIG. 4: the sealing bead of adhesive 69 between the back edge 20 and the fold line 63. The other elements of a functional fluid barrier for this type of execution—the lateral attachment areas 71—are not visible in FIG. 4. The stranded elastic area 38 of the back panel 61 provides a flexible fit of the absorbent garment 10 for the wearer and helps to form the waist opening 48. The at least one elastic strand 65 in the folded over portion of the back panel 61 helps to provide some elasticity and retraction of the back waist pocket 75; this elasticity and retraction pushes the folded over portion of the back panel 61—including the edge of the folded over portion 67—away from the skin-facing liner 44 in order to more effectively trap and contain bodily wastes.

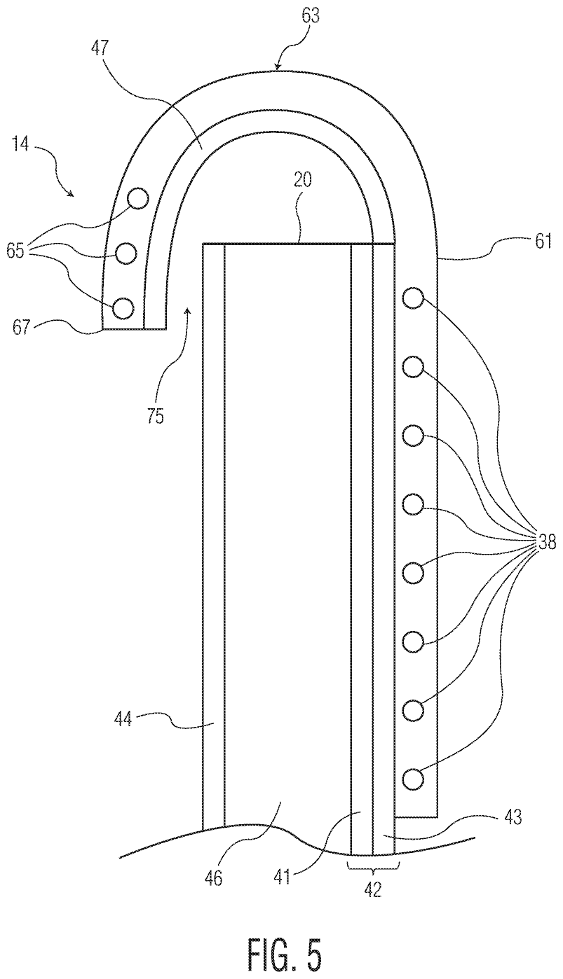

FIG. 5 depicts a different aspect of the present invention. FIG. 5 representatively shows a cross-section of the back region 14 of the absorbent garment 10, including the back panel 61 in relationship to the absorbent assembly 34 and its components: skin-facing liner 44; absorbent composite 46; and outer cover 42. As with the depiction of the invention in FIG. 4, the outer cover 42 is formed of a film layer 41 and an outer cover facing 43. The cross-section of the back region 14 in FIG. 5 shows how the back panel 61 folds over the back edge 20 of the absorbent assembly 34/absorbent composite 46. As with the execution in FIG. 4, the stranded elastic area 38 of the back panel 61 is shown as are the at least one stranded elastic 65 in the folded over portion of the back panel 61. The back waist pocket 75 is formed between the folded over portion of the back panel 61 and the skin-facing liner 44 of the absorbent assembly 34. The back waist pocket 75 is defined by the fold line 63 and the edge of the folded over portion 67. With this aspect of the invention, the fluid barrier between the folded over portion of the back panel 61 and the skin-facing liner 44 is formed by a film layer 47. The film layer 47 may be formed of materials that are similar to the materials used to form the film layer 41 of the outer cover 42. The material of the film layer 47 is desirably selected to prevent fluids from passing out of or through the back waist pocket 75.

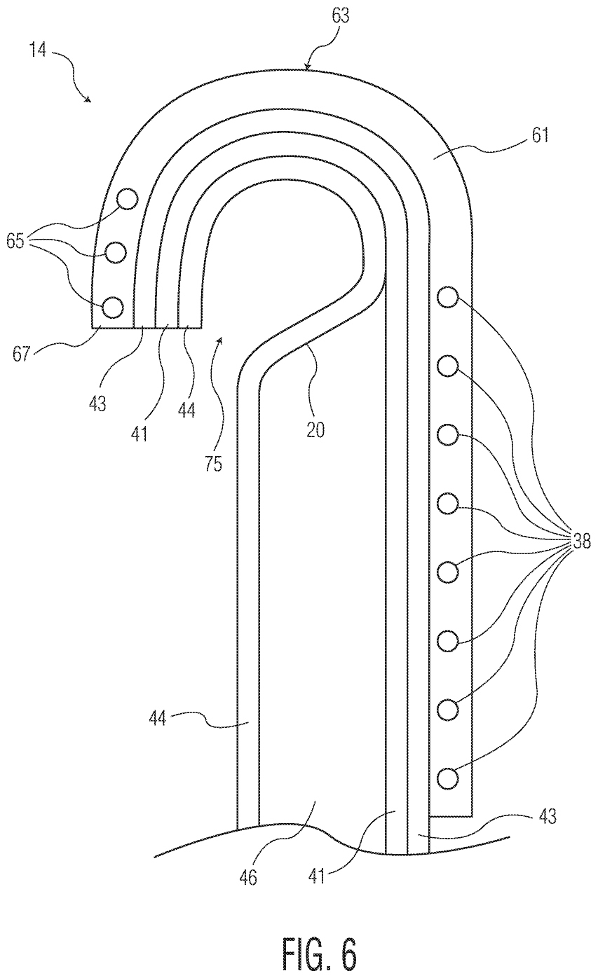

The present invention is also directed an absorbent garment 10 including a longitudinal direction 30 and a lateral direction 32, a front region 12, a back region 14 and a crotch region 16. The crotch region 16 is disposed between the front region 12 and the back region 14 and the crotch region 16 includes and absorbent assembly 34. The front region 12 includes a front panel 57 which includes an inner layer, an outer layer and a stranded elastic area 36 between the inner layer and the outer layer. The back region 14 includes a back panel 61 which includes an inner layer, an outer layer and a stranded elastic area 38 between the inner layer and the outer layer. The absorbent assembly 34 includes a skin-facing liner 44, an absorbent composite 46 and an outer cover 42. The outer cover 42 includes a liquid impermeable layer that contains fluids absorbed into the absorbent composite 46 from leaking out of the absorbent garment 10. The absorbent assembly 34 includes a front edge 18, a back edge 20 and a pair of laterally-opposed side edges 22, 24. For purposes of this aspect of the invention, the back edge 20 will refer to a back edge of the absorbent composite 46 (because the skin-facing liner 44 and the outer cover 42 extend beyond the back edge (“20”) of the absorbent composite 46). A portion of the back panel 61 is folded over the back edge 20 of the absorbent composite 46 forming a fold line 63. The folded over portion of the back panel 61 includes at least one elastic strand 65. As described above, the skin-facing liner 44 and the outer cover 42 extend in parallel with the folded over portion of the back panel 61. The front panel 57 and the back panel 61 are bonded to each other to form side seams 40 of the absorbent garment 10. FIG. 6 representatively shows a cross-section of the back region 14 of the absorbent garment 10 just described. FIG. 6 shows the absorbent assembly 34 and its components: a skin-facing liner 44; an absorbent composite 46; and an outer cover 42 (specifically, the film layer of the outer cover 41 and the outer cover facing 43 are shown). FIG. 6 also shows the back panel 61 and its stranded elastic area 38; the stranded elastic area 38—together with the stranded elastic area 36 of the front panel 57—form the waist opening 48 of the absorbent garment 10. In FIG. 6, the folded over portion of the back panel 61 is shown as is a depiction of how the skin-facing liner 44 and the outer cover (components 41 and 43) extend in parallel with the folded over portion of the back panel 61 and extend beyond the back edge 20 of the absorbent composite 46. The folded over portion of these components forms a fold line 63, terminates at an edge of the folded over portion 67 and forms a back waist pocket 75. The folded over portion of the back panel 61 includes at least one stranded elastic 65; the at least one stranded elastic 65 provides some contraction of the folded over portion so that the edge of the folded over portion 67 is lifted away from the skin-facing liner 44 over the absorbent composite 46 and so the back waist pocket 75 can be effective at trapping and containing bodily wastes. In order to improve the effectiveness of the back waist pocket 75, the edge of the folded over portion 67 does not reach the back edge 20 of the absorbent composite 46. More specifically, the distance between the edge of the folded over portion 67 and the back edge 20 is greater than five (5) millimeters (mm). With this aspect of the invention, the absorbent garment may include containment flaps (not shown in the Figures). The containment flaps may be attached to the skin-facing liner 44 of the absorbent assembly 34. The containment flaps typically extend along the length of the absorbent assembly 34 in the longitudinal direction 30. More specifically, each containment flap would extend along and proximate one of the laterally-opposed side edges 22, 24 of the absorbent assembly 34. When containment flaps are incorporated into the absorbent garment 10, the folded over portion of the back panel 61 may be attached to the un-folded portion of the skin-facing liner 44 along contact lines with each of the containment flaps. The attachment to the un-folded portion of the skin-facing liner 44 via attachment to the containment flaps may occur by adhesive or other known means of attachment.

In addition to the description of different aspects of the present invention already provided, further description of the individual components of the absorbent garment 10 is helpful. For example, the absorbent assembly 34 is configured to contain and/or absorb bodily wastes discharged from the wearer. The outer cover 42 suitably includes a material which is substantially liquid impermeable (typically, the film layer 41). The outer cover 42 can be a single layer of liquid impermeable material, but more suitably includes a multi-layered laminate structure in which at least one of the layers is liquid impermeable. For instance, the outer cover 42 can include a liquid permeable outer layer (for example, the outer cover facing 43) and a liquid impermeable inner layer (the film layer 41) that are suitably joined together by an adhesive, ultrasonic bonding, thermal bonding, pressure bonding, or combinations thereof. Suitable adhesives can be applied continuously or intermittently as beads, a spray, parallel swirls, or the like. The liquid permeable outer layer 43 can be any suitable material, including materials that provide a generally cloth-like texture. The outer layer 43 can also be made of those materials of which the liquid permeable skin-facing liner 44 is made. While it is not a necessity for the outer layer 43 to be liquid permeable, it is suitable that it provides a relatively cloth-like texture to the wearer.

The inner layer 41 of the outer cover 42 can be both liquid and vapor impermeable, or it can be liquid impermeable and vapor permeable. The inner layer 41 can be manufactured from a thin plastic film, although other flexible liquid impermeable materials can also be used. The inner layer 41, or the liquid impermeable outer cover 42 when a single layer, prevents waste material from wetting articles, such as bed sheets and clothing, as well as the wearer and caregiver.

If the outer cover 42 is a single layer of material, it can be embossed and/or matte finished to provide a more cloth-like appearance. As earlier mentioned, the liquid impermeable material can permit vapors to escape from the interior of the absorbent garment, while still preventing liquids from passing through the outer cover 42. One suitable “breathable” material is composed of a microporous polymer film or a nonwoven fabric that has been coated or otherwise treated to impart a desired level of liquid impermeability. It is also contemplated that the outer cover 42 can be stretchable, and more suitably elastic. In particular, the outer cover 42 is suitably stretchable and more suitably elastic in at least the transverse (lateral), or circumferential direction of the absorbent garment 10. In other aspects the outer cover 42 can be stretchable, and more suitably elastic, in both the transverse (lateral) 32 and the longitudinal direction 30.

The liquid permeable skin-facing liner 44 is illustrated as overlying the outer cover 42 and absorbent composite 46, and can, but need not, have the same dimensions as the outer cover 42. The skin-facing liner 44 is suitably compliant, soft feeling, and non-irritating to the wearer's skin. The skin-facing liner 44 is also sufficiently liquid permeable to permit liquid body exudates to readily penetrate through its thickness to the absorbent composite 46. Further, the skin-facing liner 44 can be less hydrophilic than the absorbent composite 46 to present a relatively dry surface to the wearer and permit liquid to readily penetrate through its thickness. The hydrophilic/hydrophobic properties can be varied across the length, width and/or depth of the skin-facing liner 44 and absorbent composite 46 to achieve the desired wetness sensation or leakage performance.

The skin-facing liner 44 can be manufactured from a wide selection of web materials, such as porous foams, reticulated foams, apertured plastic films, woven and nonwoven webs, or a combination of any such materials. For example, the skin-facing liner 44 can include a meltblown web, a spunbonded web, or a bonded-carded-web composed of natural fibers, synthetic fibers or combinations thereof. The skin-facing liner 44 can be composed of a substantially hydrophobic material, and the hydrophobic material can optionally be treated with a surfactant or otherwise processed to impart a desired level of wettability and hydrophilicity. The surfactant can be applied by any conventional means, such as spraying, printing, brush coating or the like. The surfactant can be applied to the entire skin-facing liner 44 or can be selectively applied to particular sections of the skin-facing liner 44, such as the medial section along the longitudinal center line.

The skin-facing liner 44 can also be stretchable, and, more suitably, it can be elastomeric. In particular, the skin-facing liner 44 is suitably stretchable and more suitably elastomeric in at least the lateral or circumferential direction 32 of the absorbent garment 10. In other aspects, the skin-facing liner 44 can be stretchable, and more suitably elastomeric, in both the lateral direction 32 and the longitudinal direction 30.

The absorbent composite 46 is suitably compressible, conformable, non-irritating to a wearer's skin, and capable of absorbing and retaining liquids and certain body wastes. For example, the absorbent composite 46 can include cellulosic fibers (e.g., wood pulp fibers), other natural fibers, synthetic fibers, woven or nonwoven sheets, scrim netting or other stabilizing structures, superabsorbent material, binder materials, surfactants, selected hydrophobic materials, pigments, lotions, odor control agents or the like, as well as combinations thereof.

The materials can be formed into a web structure by employing various conventional methods and techniques. For example, the absorbent composite 46 can be formed by a dry-forming technique, an air forming technique, a wet-forming technique, a foam-forming technique, or the like, as well as combinations thereof. Furthermore, the absorbent composite 46 can itself encompass multiple layers in a Z-direction (e.g., thickness) of the absorbent composite 46. Such multiple layers can take advantage of differences in absorbent capacity, such as by placing a lower absorbent capacity material layer closer to the skin-facing liner 44 and a higher absorbent capacity material closer to the outer cover 42. Likewise, discrete portions of a single-layered absorbent composite can encompass higher capacity absorbents, and other discrete portions of the composite can encompass lower capacity absorbents.

Superabsorbent material is suitably present in the absorbent composite 46 in an amount of from about 0 to about 100 weight percent based on total weight of the absorbent composite 46. The absorbent composite 46 can suitably have a density within the range of about 0.10 to about 0.60 grams per cubic centimeter. Superabsorbent materials are well known in the art and can be selected from natural, synthetic, and modified natural polymers and materials. The superabsorbent materials can be inorganic materials, such as silica gels, or organic compounds, such as crosslinked polymers. Typically, a superabsorbent material is capable of absorbing at least about 10 times its weight in liquid, and preferably is capable of absorbing more than about 25 times its weight in liquid.

The absorbent composite 46 can alternatively include a coform material. The term “coform material” generally refers to composite materials including a mixture or stabilized matrix of thermoplastic fibers and a second non-thermoplastic material. As an example, coform materials are made by a process in which at least one meltblown die head is arranged near a chute through which other materials are added to the web while it is forming. Such other materials can include, but are not limited to, fibrous organic materials such as woody or non-woody pulp such as cotton, rayon, recycled paper, pulp fluff and also superabsorbent particles, inorganic absorbent materials, treated polymeric staple fibers and the like. Any of a variety of synthetic polymers can be utilized as the melt-spun component of the coform material. For instance, in certain aspects, thermoplastic polymers can be utilized. Some examples of suitable thermoplastics that can be utilized include polyolefins, such as polyethylene, polypropylene, polybutylene and the like; polyamides; and polyesters. In one aspect, the thermoplastic polymer is polypropylene.

In one suitable aspect, the absorbent composite 46 is stretchable so as not to inhibit the stretchability of other components to which the absorbent composite 46 can be adhered, such as the outer cover 42 and the skin-facing liner 44. After being formed or cut to a desired shape, the absorbent composite 46 can be wrapped or encompassed by a suitable wrap (not shown) that aids in maintaining the integrity and shape of the absorbent composite 46.

The absorbent assembly 34 can also include a surge management layer (not shown) located adjacent the absorbent composite 46 (e.g., between the absorbent composite 46 and the skin-facing liner 44) to help decelerate and diffuse surges or gushes of liquid that can be rapidly introduced into the absorbent composite 46 of the absorbent garment 10 by the wearer. Desirably, the surge management layer can rapidly accept and temporarily hold the liquid prior to releasing the liquid into the storage or retention portions of the absorbent composite 46. Examples of suitable surge management layers are described in U.S. Pat. No. 5,486,166 issued Jan. 23, 1996 to Bishop et al.; U.S. Pat. No. 5,490,846 issued Feb. 13, 1996 to Ellis et al.; and U.S. Pat. No. 5,820,973 issued Oct. 13, 1998 to Dodge, II et al., the entire disclosures of which are hereby incorporated by reference.

As already described in relation to the various aspects of the present invention, the absorbent assembly 34 can include a pair of containment flaps (not shown) that extend longitudinally along the absorbent assembly 34 and are adapted to provide a barrier to the lateral flow of body exudates as is known in the art. The containment flaps can be connected to the skin-facing liner 44 or other components of the absorbent assembly 34. Suitable configurations of the containment flaps are described, for example, in U.S. Pat. No. 5,599,338 issued Feb. 4, 1997, to K. Enloe, the entirety of which is incorporated herein by reference.

The front panel 57 of the absorbent garment 10 includes a multi-layer construction including an inner or body-facing layer, an outer or garment-facing layer, and a stranded elastic area 36 disposed between the inner layer and the outer layer. The inner layer and the outer layer each include, respectively, a body-facing side and a garment-facing side. The garment-facing side of the inner layer is connected to the body-facing side of the outer layer by suitable means such as adhesives, ultrasonic bonds, thermal bonds, pressure bonds, or combinations thereof. Typically, a portion of the outer layer is folded over a top edge of the inner layer. The folded over portion of the outer layer is connected to the body-facing side of the inner layer to close the top edge of the waist opening 48 (FIG. 2), and enclose the stranded elastic area 36 between the inner layer and the outer layer.

The inner layer is typically constructed from a non-apertured nonwoven material. Suitable nonwovens include single layer nonwovens, such as spunbond webs, and nonwoven laminates. In one suitable aspect, the inner layer includes a spunbond/spunbond/spunbond (“SSS”) laminate. In another suitable aspect, the inner layer includes at least one meltblown layer positioned between two or more spunbond layers to form a spunbond/meltblown/spunbond (“SMS”) laminate. In one particular aspect, the inner layer includes a spunbond/spunbond/meltblown/meltblown/spunbond laminate (“SSMMS”). The nonwoven laminate can have other configurations and possess any desired number of meltblown and spunbond layers, such as spunbond/meltblown/meltblown/spunbond laminates (“SMMS”), spunbond/meltblown laminates (“SM”), etc. In addition to or as an alternative to meltblown and spunbond webs, a variety of other nonwoven webs can also be used to form the inner layer including, for example and without limitation, through-air bonded carded webs, thermally bonded carded webs, wet-laid webs, coform webs, and hydraulically entangled webs.

The inner layer may be liquid-impermeable and vapor permeable. That is, the inner layer permits vapors to escape from the interior of the absorbent garment, while still preventing liquids from passing through the inner layer. The inner layer can be treated or otherwise processed to impart a desired level of liquid impermeability to the inner layer.

The stranded elastic area 36 is attached to at least one of the inner layer and the outer layer to impart a desired level of elasticity to the front panel 57. The stranded elastic area 36 may be attached to the inner layer and/or the outer layer by any suitable means including, for example, adhesives. The stranded elastic area 36 may be stretched and then adhered to one or both of the inner layer and the outer layer, or adhered to one or both of the inner layer and the outer layer when the layers are in a gathered state to impart a desired level of elasticity to the front panel 57. In other aspects, the stranded elastic area 36 may be adhered to one or both of the inner layer and the outer layer, and then elasticized or shrunk, for example with the application of heat, such that elastic retractive forces are imparted to the front panel 57.

The stranded elastic area 36 may be formed of a variety of suitable elastic materials, including strands or ribbons of natural rubber, synthetic rubber, or thermoplastic elastomeric polymers. In the aspect shown in FIG. 1, the stranded elastic area 36 includes a plurality of elastomeric strands extending in the lateral direction 32 (FIG. 1) or circumferential direction (FIG. 2) of the absorbent garment 10. In one suitable aspect, the elastomeric strands include dry-spun coalesced multifilament spandex elastomeric strands sold under the trade name LYCRA® and available from Invista of Wichita, Kans., U.S.A. Other suitable materials from which the stranded elastic area 36 may be constructed include vertical filament laminate (VFL) materials, an example of which is described in U.S. Pat. No. 6,916,750 to Thomas et al., which is hereby incorporated by reference.

The outer layer is vapor permeable, and may be liquid permeable or liquid impermeable. The outer layer may be constructed from an apertured nonwoven, such as a single layer nonwoven or a nonwoven laminate. In one suitable aspect, the outer layer includes a spunbond/spunbond/spunbond (“SSS”) laminate. In another suitable aspect, the outer layer includes at least one meltblown layer positioned between two or more spunbond layers to form a spunbond/meltblown/spunbond (“SMS”) laminate. In one aspect, the outer layer includes a spunbond/spunbond/meltblown/meltblown/spunbond laminate (“SSMMS”). The nonwoven laminate can have other configurations and possess any desired number of meltblown and spunbond layers, such as spunbond/meltblown/meltblown/spunbond laminates (“SMMS”), spunbond/meltblown laminates (“SM”), etc. In addition to or as an alternative to meltblown and spunbond webs, a variety of other nonwoven webs can also be used to form the outer layer including, for example and without limitation, through-air bonded carded webs, thermally bonded carded webs, wet-laid webs, coform webs, and hydraulically entangled webs.

The inner layer and the outer layer are joined together in face-to-face relationship by suitable means such as adhesives, ultrasonic bonding, thermal bonding, pressure bonding, or combinations thereof. Suitable adhesives, which can be applied continuously or intermittently as beads, a spray, parallel swirls, or the like, include elastomeric adhesives (i.e. materials capable of at least 75% elongation without rupture), such as aqueous-based styrene butadiene adhesives, neoprene, polyvinyl chloride, vinyl copolymers, polyamides, and ethylene vinyl terpolymers.

The structure used to form the back panel 61 of the absorbent garment 10 has the same construction and configuration as the front panel 57. That is, the back panel 61 includes a multi-layer construction including an inner or body-facing layer, an outer or garment-facing layer, and a stranded elastic area 65 disposed between the inner layer and the outer layer. The inner layer and the outer layer each include, respectively, a body-facing side and a garment-facing side. As described herein, a portion of the back panel is folded over the back edge 20 of the absorbent assembly 34/absorbent composite 46. The folded over portion of the back panel 61 also closes a top edge of the waist opening 48 (FIG. 2), and encloses the stranded elastic area 65 between the inner layer and the outer layer. The layers of the back panel 61 may be constructed of the same materials as the layers of the front panel 57 described above.

In the interests of brevity and conciseness, any ranges of values set forth in this disclosure contemplate all values within the range and are to be construed as support for claims reciting any sub-ranges having endpoints that are whole number values within the specified range in question. By way of hypothetical example, a disclosure of a range of from 1 to 5 shall be considered to support claims to any of the following ranges: 1 to 5; 1 to 4; 1 to 3; 1 to 2; 2 to 5; 2 to 4; 2 to 3; 3 to 5; 3 to 4; and 4 to 5.

The dimensions and values disclosed herein are not to be understood as being strictly limited to the exact numerical values recited. Instead, unless otherwise specified, each such dimension is intended to mean both the recited value and a functionally equivalent range surrounding that value. For example, a dimension disclosed as “40 mm” is intended to mean “about 40 mm.”

All documents cited in the Detailed Description are, in relevant part, incorporated herein by reference; the citation of any document is not to be construed as an admission that it is prior art with respect to the present disclosure. To the extent that any meaning or definition of a term in this written document conflicts with any meaning or definition of the term in a document incorporated by references, the meaning or definition assigned to the term in this written document shall govern.

While particular aspects of the present disclosure have been illustrated and described, it would be obvious to those skilled in the art that various other changes and modifications can be made without departing from the spirit and scope of the disclosure. It is therefore intended to cover in the appended claims all such changes and modifications that are within the scope of this disclosure.

We claim:

1. An absorbent garment comprising:
a longitudinal direction and a lateral direction;
a front region, a back region, a crotch region, the crotch region being disposed between the front region and the back region, and the crotch region including an absorbent assembly;
a front panel in the front region, wherein the front panel includes an inner layer, an outer layer and a stranded elastic area disposed between the inner layer and the outer layer;
a back panel in the back region, wherein the back panel includes an inner layer, an outer layer and a stranded elastic area disposed between the inner layer and the outer layer;
the absorbent assembly including a skin-facing liner, an absorbent composite and an outer cover, wherein the outer cover includes a liquid impermeable layer; the absorbent assembly also includes a front edge, a back edge and a pair of laterally-opposed side edges;
wherein a portion of the back panel is folded over the back edge of the absorbent assembly forming a fold line, wherein the folded over portion of the back panel includes at least one elastic strand and there is a fluid barrier between the folded over portion and the skin-facing liner of the absorbent assembly; and
the front panel and the back panel are bonded to each other to form side seams of the absorbent garment.

2. The garment of claim 1, wherein the folded over portion of the back panel is attached to the skin-facing liner of the absorbent assembly proximate and along each of the laterally-opposed side edges of the absorbent assembly.

3. The garment of claim 1, wherein the fluid barrier is a bead of adhesive between the folded over portion of the back panel and the skin-facing liner along the back edge of the absorbent composite.

4. The garment of claim 1, wherein the fluid barrier is a film layer attached to the folded over portion of the back panel.

5. The garment of claim 2, wherein the absorbent assembly further includes a pair of containment flaps that extend along a length of the absorbent assembly in the longitudinal direction and each containment flap extends along one of the laterally-opposed side edges of the absorbent assembly; and wherein the folded over portion of the back panel is attached to the skin-facing liner of the absorbent assembly along contact lines with each of the containment flaps.

6. The garment of claim 5, wherein the folded over portion of the back panel is attached to each of the containment flaps by adhesive.

7. An absorbent garment comprising:
a longitudinal direction and a lateral direction;
a front region, a back region, a crotch region, the crotch region being disposed between the front region and the back region, and the crotch region including an absorbent assembly;
a front panel in the front region, wherein the front panel includes an inner layer, an outer layer and a stranded elastic area disposed between the inner layer and the outer layer;
a back panel in the back region, wherein the back panel includes an inner layer, an outer layer and a stranded elastic area disposed between the inner layer and the outer layer;
the absorbent assembly including a skin-facing liner, an absorbent composite and an outer cover, wherein the outer cover includes a liquid impermeable layer; the absorbent assembly also includes a front edge and a pair of laterally-opposed side edges and further wherein the skin-facing liner and the outer cover extend beyond a back edge of the absorbent composite;
wherein a portion of the back panel is folded over the back edge of the absorbent composite forming a fold line, wherein the folded over portion of the back panel includes at least one elastic strand and the skin-facing liner and the outer cover extend in parallel with the folded over portion of the back panel; and
the front panel and the back panel are bonded to each other to form side seams of the absorbent garment.

8. The garment of claim 7, wherein the folded over portion of the back panel extending in parallel with the skin-facing liner and the outer cover end at an edge of the folded over portion.

9. The garment of claim 8, wherein the edge of the folded over portion does not reach the back edge of the absorbent composite.

10. The garment of claim 9, wherein the distance between the edge of the folded over portion and the back edge of the absorbent composite is greater than 5 millimeters.

11. The garment of claim 7, wherein the absorbent assembly further includes a pair of containment flaps that extend along a length of the absorbent assembly in the longitudinal direction and each containment flap extends along a laterally-opposed side edges of the absorbent assembly; and wherein the folded over portion of the back panel is attached to the skin-facing liner of the absorbent assembly along contact lines with each of the containment flaps.

12. The garment of claim 11, wherein the folded over portion of the back panel is attached to each of the containment flaps by adhesive.
